(19) United States
(12) Patent Application Publication
(10) Pub. No.: US 2009/0118349 A1
(43) Pub. Date: May 7, 2009

(54) BIS-ALKYLATING AGENTS AND THEIR USE IN CANCER THERAPY

(75) Inventors: Zoltan Szekely, Piscataway, NJ (US); Matthew Gerard McDonnell, Piscataway, NJ (US)

Correspondence Address: Matthew McDonnell, 61 Ross Hall Blvd. South, Piscataway, NJ, 08854, US

(73) Assignee: ONCO-PHARMAKON INCORPORATED, Piscataway, NJ (US)

(21) Appl. No.: 12/192,990

(22) Filed: Aug. 16, 2008

Related U.S. Application Data
(63) Continuation of application No. 60/956,384, filed on Aug. 16, 2007.

(51) Int. Cl. A61K 31/409; A61K 31/403; A61K 31/407

(52) U.S. Cl. 514/411

(57) ABSTRACT

The present invention relates to (i) conjugates comprising two DNA alkylating subunits linked by a moiety fitting to the minor groove of the DNA, (ii) to their preparation and (iii) to their use in cancer therapy. The alkylating subunits are especially cytotoxic under hypoxic conditions found in cancer cells. The compounds of the present invention and compositions thereof are useful in the treatment of cancer in a mammal, both alone or in a combination with other anti-cancer agents (e.g. checkpoint abrogators) and/or radiation. They may also be used as cytotoxic units for gene-directed enzyme-prodrug therapy (GDEPT) and antibody-directed enzyme-prodrug therapy (ADEPT).

The present invention provides the compounds of Formula (I), Formula (II) and Formula (III):

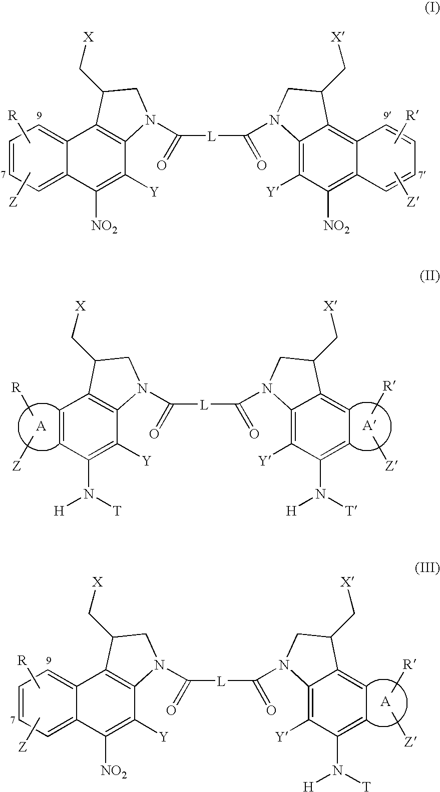

for treating cancer in a mammal.

### CROSS-REFERENCE TO RELATED APPLICATIONS

1. Unites States patent, U.S. Pat. No. 5,541,339 Kelly, R. C., Aristoff, P. A., CC-1065 ANALOGS HAVING TWO CPI SUBUNITS

2. International Patent, International Publication Number: WO 2006/043839 A1 Denny, W. A., Wilson, W. R., Stevenson, R. J., Tercel, M., Atwell, G. J., Yang, S., Patterson, A. V., Pruijn, F. B., NITROBENZINDOLES AND THEIR USE IN CANCER THERAPY

3. International Patent, International Publication Number: WO 2006/034266 A2 Lin, X., King, I., Belcourt, M. F., Doyle, T. W., PHOSPHATE-BEARING PRODRUGS OF SULFONYL HYDRAZINES AS HYPOXIA-SELECTIVE ANTINEOPLASTIC AGENTS

### STATEMENT REGARDING FEDERALLY SPONSORED RESEARCH OR DEVELOPMENT

Not Applicable

### REFERENCE TO A SEQUENCE LISTING, A TABLE OR A COMPUTER PROGRAM, LISTING COMPACT DISC APPENDIX

Not Applicable

### TECHNICAL FIELD

The present invention relates to conjugates comprised of two DNA alkylating subunits linked by a moiety fitting to the minor groove of the DNA. These alkylating subunits are activated under the hypoxic condition which is found in various cancer cells. Compounds, which consist of two alkylating subunits linked together, result in (i) high anti-cancer activity and (ii) improved hypoxic/oxic cytotoxicity ratio. The compounds of the present invention and compositions thereof are useful in the treatment of cancer in a mammal both alone or in a combination with other anti-cancer agents (e.g. check point abrogators) and/or radiation or as cytotoxic molecules for gene-directed enzyme-prodrug therapy (GDEPT) and antibody-directed enzyme-prodrug therapy (ADEPT).

### BACKGROUND OF THE INVENTION

Solid tumors contain regions of cells characterized by low oxygen concentration. This phenomena (know as hypoxia) is a result of the inefficient microvasculature system of the tumor (Brown, M. J. and Wilson, W. R. Nature Reviews, Cancer, 2004, 4, 437-447). Hypoxia occurs in most tumors. Extreme hypoxia levels have been noted in tumors smaller than 1 mm in diameter. As tumors grow beyond this size the levels of hypoxia in the tumor vary as vasculature development balances with tumor growth. (Li, X. F. et al., Cancer Research, 2007, 67, 7646-7653 and Li, X. F. and J. A. O'Donoghue, Cancer Letters, 2008, 246, 172-180). In recent years hypoxia has became a frequently targeted condition in anti-cancer drug design and development (Denny, W. A. Curr. Med. Chem.—Anti-cancer Agents, 2004, 4, 395-399). Hypoxia-sensitive drugs include various classes of compounds activated by either endogenous nitroreductases or by the exogenous enzymes delivered using ADEPT or GDEPT technologies.

In this invention we describe a family of compounds, which target DNA in cancerous cells that exhibit hypoxic conditions. The major advantage of such an approach is to minimize the systemic cytotoxicity (i.e. side effects) while providing sufficient cytotoxicity within the hypoxic tumors.

As an additional benefit of targeting hypoxia, a certain percentage of the activated molecules will target adjacent cells. This “by-stander” effect could increase the anti-tumor activity of the drug in vivo by impacting more malignant cells in close proximity as well (Wilson, W. R. et al. Radiat. Res. 2007, 167, 625-636).

We have chosen the CC-1065 antitumor antibiotic (the first member of the cyclopropyl-indole containing natural product family, FIG. 1) as the lead structural element for our compounds to provide the appropriate level of cytotoxicity. The in-depth investigation of CC-1065 (Boger, D. L. and Garbaccio, R. M. Acc. Chem. Res. 1999, 32, 1043-1052) led to the syntheses of numerous new cytotoxic variants (CBI, CBA etc. FIG. 2) as well as three clinical drug candidates: Adozelesin, Carzelesin and Bizelesin (FIG. 3). While Adozelesin and Carzelesin are mono-alkylating agents, Bizelesin (Kelly, C. R., Aristoff, P. A., U.S. Pat. No. 5,541,339) is a bis-alkylating molecule, consisting two CPI units (CC-1065 pharmacophore) linked together by a bis-indole subunit. Since bizelesin is the only crosslinking agent in this group, this drug had shown superior anti-cancer activity in vivo to its mono-alkylating analogues (Pitot, H. C. et al. Clin. Cancer Res. 2002, 8, 712-717). Later cell biology studies on Bizelesin revealed (Cao, P. et al. Mol. Cancer. Therapeutics, 2003, 2, 651-659) a unique mechanism of action being responsible for its outstanding potency. The scope of the invention presented here is to identify a group of compounds which act like bis-alkylating agents but are activated only under hypoxic conditions.

In order to change regular cyclopropyl indol-based mono-alkylating agents to hypoxia-sensitive prodrugs (i.e. forming an active drug from a prodrug only under hypoxic conditions), there are three approaches published and depicted on FIG. 4.

(i) by changing the phenolic hydroxyl group to an amino group the new (e.g. CBA) derivatives can be labeled by a hypoxia-sensitive group, typically nitro-aromatic carbamate, this allows bio-reduction to occur releasing the toxic amino form of the drug,

(ii) as in the previously described approach, but using a more stable nitro-aromatic masking group and reducing them by E. coli NTR enzyme delivered using ADEPT or GDEPT methods (Hay, M. P. et al, J. Med. Chem. 2003, 46, 5533-5545) and

(iii) the final means is to change the electronic structure of the indole subunit by electron withdrawing groups (e.g. sulfamoyl), and replacing the amino by a nitro group, the resulted structure can be reduced by endogenous enzymes forming the highly toxic amino form of the drug (Denny, W. A. et al., International Patent, 2006, WO 2006/043839).

As a fundamental change to the approaches described above, we present a family of bis-alkylating compounds suitable for bio-reduction by endogenous enzymes under hypoxic conditions. This approach represents a significant synergy of high toxicity and high selectivity in cancer chemotherapy.

### BRIEF SUMMARY OF THE INVENTION

The present invention provides the compounds of Formula I, Formula II and Formula III,

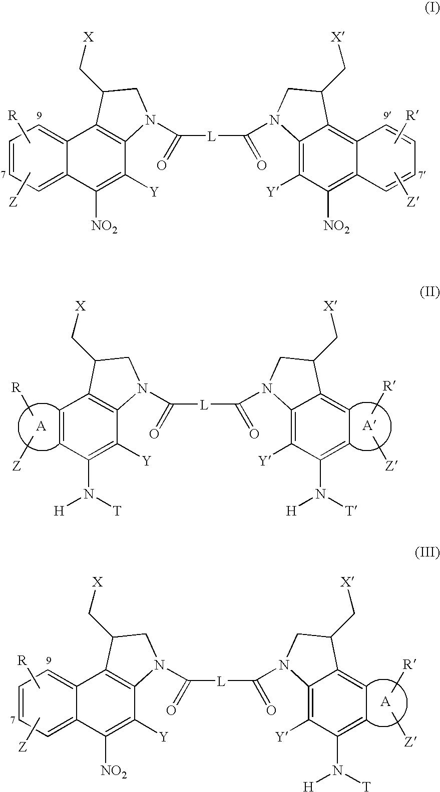

Wherein X and X′ are independently selected from halogen (preferably chlorine or bromine), Wherein R, R′, Y, Y′, Z and Z′ are independently selected from hydrogen, halogen, C₁₋₄ alkyl, OR¹, OP(O)(OH)₂, NR¹C(O)OR², OC(O)R¹, NHC(O)NR¹R², SR¹, NR¹₂, COR¹, SOR₁, SO₂R¹, SO₂NH₂, SO₂NR¹OR², SO₂NR¹NR²₃, SO₂NHCOR¹, CO₂R¹, CONR¹₂, CONHSO₂R¹, CF₃, CN, NO₂, where R¹ and R² represent hydrogen or C₁₋₄ alkyl, where R, R′, Z and Z′ are located at any of the available positions 6-9 or located at any of the available positions of the A and A′ ring systems and R, R′, Z and Z′ independently represent a C₁₋₄ alky group or a SO₂NHR¹ group optionally further substituted with one or more amino, thiol, hydroxyl groups, each amino or thiol group being further optionally substituted with one or two C₁₋₄ alkyl groups and each hydroxyl group being further optionally substituted with a phosphate group,
wherein A and A′ are independently selected aromatic, heteroaromatic or saturated carbocyclic or heterocyclic systems (preferably one of the following rings: benzene, pyridine, imidazole, pyrrole, the saturated part of teralin), or A and A′ independently represent two hydrogen atoms, wherein L represent a linker selected from but not limited to: urea, carbamate, ethylene glycol, propylene glycol, unfused aromatic, fused aromatic, hetero-aromatic, 1,4-phenylenediacryloyl, 1,3-phenylenediacryloyl building blocks, with a special interest in linked indoles like N,N-Bis[indole-5-yl-(2-hydroxyethyl)]methylamine,
and wherein T and T′ represent a hypoxia-sensitive group, independently selected from nitro-group containing aromatic groups (e.g. 4-nitro-benzyl) with optional substituents (like one or more amino, thiol, hydroxyl groups), with optional further substituents (like phosphate or C₁₋₄ alkyl groups).

The formulae represent 3 combinations of hypoxia activated drugs, CBN-CBN, CBA(T)-CBA(T) and CBN-CBA(T) after activation by bio-reduction all of them form CBA-CBA type molecules (FIG. 4).

### BRIEF DESCRIPTION OF THE SEVERAL VIEWS OF THE DRAWINGS

FIG. 1 depicts the structures of the natural products discussed in the background of the invention.

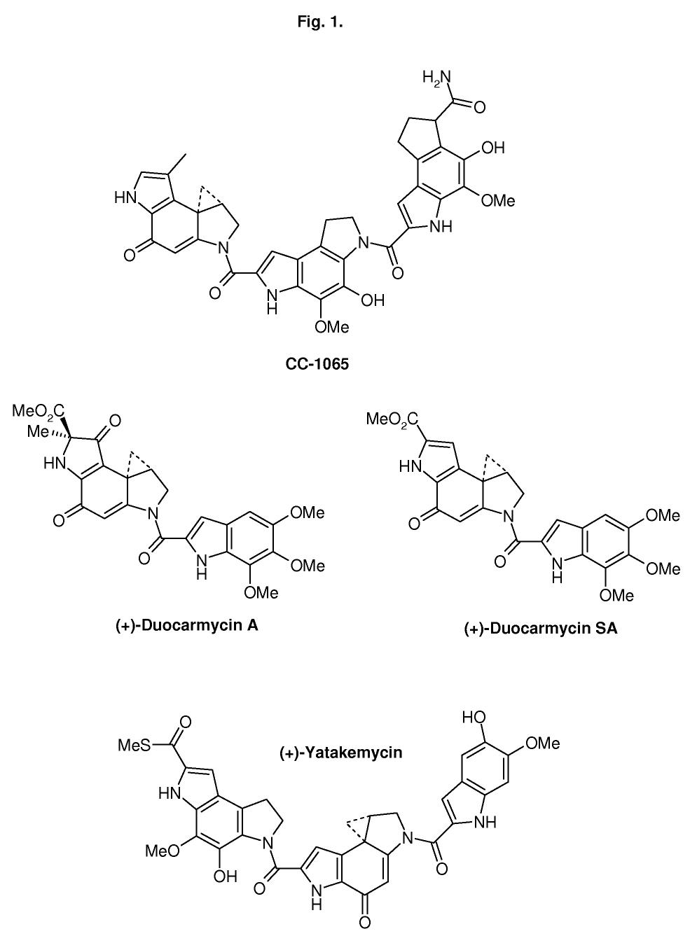

FIG. 2 depicts the synthetic analogues derived from the cytotoxic unit of the CC-1065 anti-tumor antibiotics.

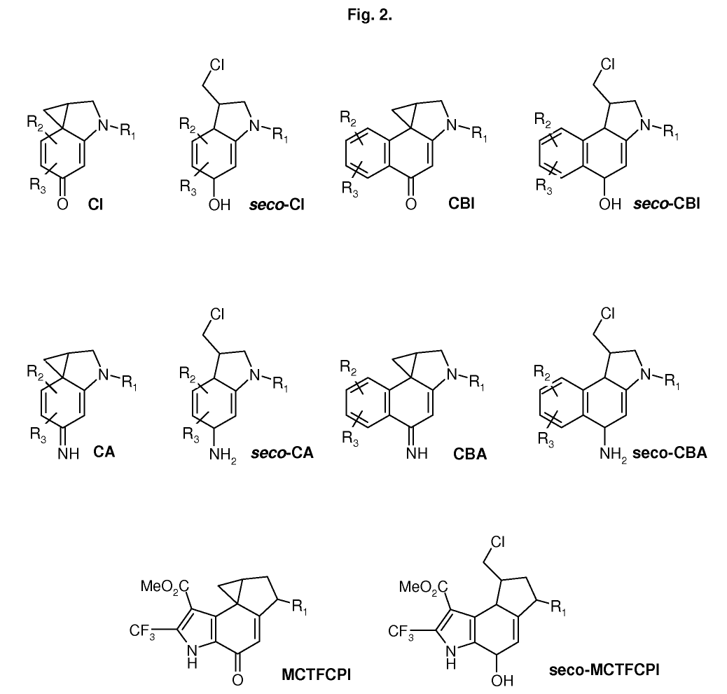

FIG. 3 depicts the structures of the drug candidates in recent clinical trials bearing cyclopropyl indole moieties.

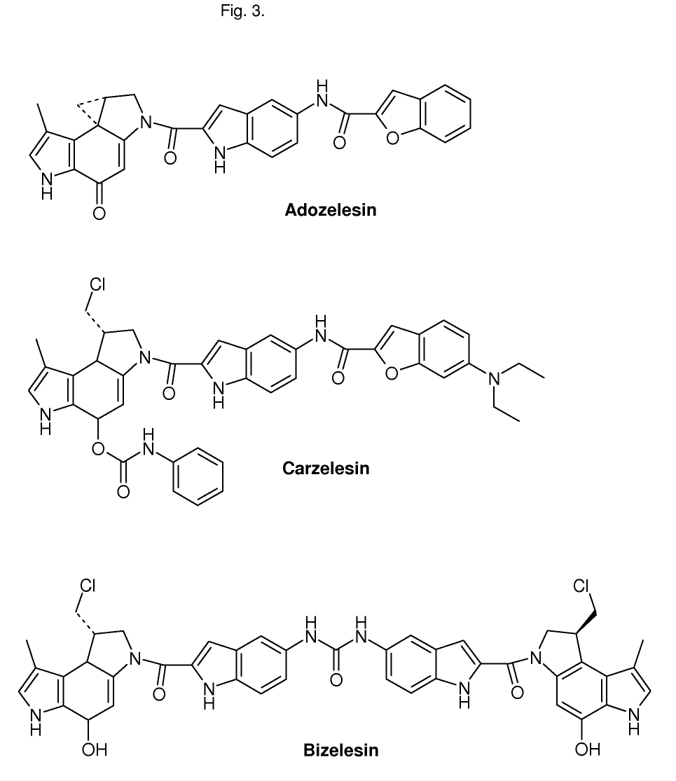

FIG. 4 is a schematic representation of the bio-reductive pathways activating the new compounds described in the invention.

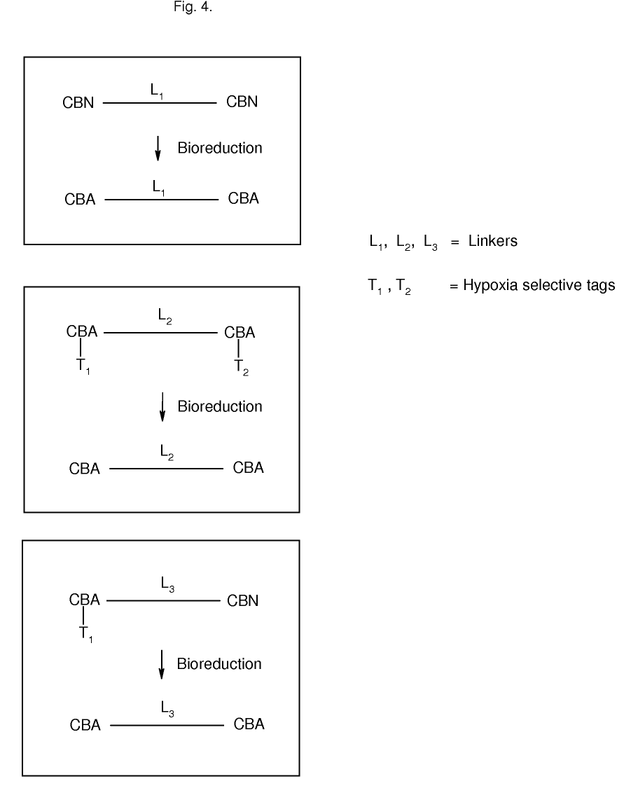

FIG. 5 is a drawing representing the nucleofil substitution of the sulfonyl chloride and the subsequent de-blocking of the trifluoroacetyl protecting group.

FIG. 6 is a drawing representing the synthesis of one of the new linkers.

FIG. 7 depicts examples of racemic mixtures as well as optically pure compounds of this invention.

FIG. 8 depicts the coupling of the new linker with two CBN derivatives.

FIG. 9 depicts the synthetic scheme of 1,3-Bis-[2-(1-chloromethyl-5-nitro-7-sulfamoyl-1,2-dihydro-benzo[e]indole-3-carbonyl)-benzofuran-5-yl]-urea.

FIG. 10 depicts the 1,3-Bis-{2-[1-chloromethyl-5-(3-dihydrogenphosphonoxy-4-nitrobenzyloxycarbonyl-amino)-1,2-dihydro-benzo[e]indole-3-carbonyl]-1H-indol-5-yl}-urea.

The following abbreviations are used herein:
EDC Ethyl-dimethylaminopropyl-carbodiimide hydrochloride
Tos-OH p-Toluenesulfonic acid
DMA N,N-Dimethylacetamide
HOAt 1-Hydroxy-7-azabenzotriazole
DMAP 4-Dimethylaminopyridine
Py Pyridine
2,6-lutidine 2,6-Dimethylpyridine
Grubbs' Carbene Benzylidene-bis(tricyclohexylphosphine)dichlororuthenium
Ar—C(O)Cl various aromatic or benzylic chloroformate
TMSBr Trimethylsilyl bromide
K₂CO₃Potassium carbonate
DEAD Diethylazodicarboxylate
PPH3 Triphenylphosphine
NaOH Sodium hydroxide
THF Tetrahydrofuran
MeOH Methanol
(COCl)₂ Oxalyl chloride
TFFH Fluoro-N,N,N′,N′-teramethylformamidinium hexafluorophosphate

### DETAILED DESCRIPTION OF THE INVENTION

The present invention also provides compositions, including pharmaceutical compositions, comprising the conjugate of formulae (I), (II) and (III) or combinations thereof, and a carrier, alone or in further combination with other active agents, such as adjuvants and anti-cancer agents. Preferably, the pharmaceutical compositions further comprise a pharmaceutically acceptable carrier.

Also provided is a method of inhibiting gene expression in a mammal by administering to a mammal a gene expression-inhibiting effective amount of the conjugate of formulae (I), (II), and (III) or combinations thereof, and a carrier, alone or in further combination with other active agents. Preferably, the pharmaceutical compositions further comprise a pharmaceutically acceptable carrier.

One ordinarily skilled in the art will appreciate that suitable methods of administering a conjugate or composition thereof to a mammal, such as a human, are known, and, although more than one route can be used to administer a particular composition, a particular route can provide a more immediate and more effective reaction than another route. If the cancer is in the form of a tumor, preferably the conjugate or composition thereof is administered intratumorally or peritumorally. Pharmaceutically acceptable carriers are also well-known in the art. The choice of carrier will be determined, in part, by the particular conjugate or composition and by the particular method used to administer the composition. Accordingly, there are a wide variety of suitable formulations of the pharmaceutical compositions of the present invention.

Formulations suitable for oral administration can consist of (a) liquid solutions, such as an effective amount of the conjugate of any of formulae (I), (II) and (III) dissolved in diluents, such as water or saline, (b) capsules, sachets or tablets, each containing a predetermined amount of the active ingredient, as solids or granules, (c) suspensions in an appropriate liquid, and (d) suitable emulsions.

Tablet forms can include one or more of lactose, mannitol, cornstarch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, such carriers as are known in the art.

The conjugates of the present invention, alone or in combination with other suitable components, can be made into aerosol formulations to be administered via inhalation. These aerosol formulations can be placed into pressurized acceptable propellants, such as dichlorodifluoromethane, hydrofluorocarbon (such as HFC 134a and/or 227), propane, nitrogen and the like. The conjugates of the present invention, alone or in combination with other suitable components, can be made into creams or transdermal patches for topical application.

The conjugates of the present invention, alone or in combination with other suitable components, can be made into mechanical devices such as stints or sub-dermal implants.

Formulations suitable for parenteral administration include aqueous and non-aqueous solutions, isotonic sterile injection solutions, which can contain anti-oxidants, buffers, bacteriostats and solutes that render the formulation isotonic with the blood of the intended recipient, and aqueous and non-aqueous sterile suspensions that can include suspending agents, solubilizers, thickening agents, stabilizers and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The dose administered to a mammal, particularly a human, in the context of the present invention should be sufficient to effect a prophylactic or therapeutic response in the mammal over a reasonable time frame. The dose will be determined by the strength of the particular composition employed (taking into consideration, at least, the bioactivity of any decomposition products derived from the conjugates) and the condition of the mammal (e.g., human), as well as the body weight of the mammal (e.g., human) to be treated. The size of the dose also will be determined by the existence, nature, and extent of any adverse side effects that might accompany the administration of a particular composition. A suitable dosage for internal administration is 0.00001 to 100 mg/kg of body weight per day, such as 0.01 to 35 mg/kg of body weight per day or 0.05 to 5 mg/kg of body weight per day. A suitable concentration of the conjugate in pharmaceutical compositions for topical administration is 0.0005 to 1% (by weight), preferably 0.002 to 0.5%.

The conjugates of any of formulae (I), (II) and (III) or compositions thereof are useful for treating a mammal, such as a human, for cancer. The method comprises administering to the mammal, e.g., human, a cancer-inhibiting effective amount of a conjugate of any of formulae (I), (II) and (III) or a composition thereof, whereupon the mammal is treated for cancer. The treatment can be prophylactic or therapeutic. By “prophylactic” is meant any degree in inhibition of the onset of cancer, including complete inhibition. By “therapeutic” is meant any degree in inhibition of the growth or metastasis of the cancer in the mammal (e.g., human).

The method can be used in combination with other known treatment methods, such as radiation, surgery, or the administration of other active agents, such as adjuvants or other anti-cancer agents and their prodrugs. The combinational therapy can include G2 check point abrogators and any other compound which can alter the cell cycle of tumor cells. Examples of cyotoxic agents and their prodrugs include genistein, okadaic acid, 1-β-D-arabinofuranosyl-cytosine, arabinofuranosyl-5-aza-cytosine, cisplatin, carboplatin, actinomycin D, asparaginase, bis-chloro-ethyl-nitroso-urea, bleomycin, chlorambucil, cyclohexyl-chloro-ethyl-nitroso-urea, cytosine arabinoside, daunomycin, etoposide, hydroxyurea, melphalan, mercaptopurine, mitomycin C, nitrogen mustard, procarbazine, teniposide, thioguanine, thiotepa, vincristine, 5-fluorouracil, 5-fluorocytosine, adriamycin, cyclophosphamide, methotrexate, vinblastine, doxorubicin, leucovorin, taxol, anti-estrogen agents such as tamoxifen, intracellular antibodies against oncogenes, the flavonol quercetin, Guan-mu-tong extract, retinoids such as fenretinide, nontoxid retinoid analogues such as N-(4-hydroxyphenyl)-retinamide (HPR), and monoterpenes such as limonene, perillyl alcohol and sobrerol.

The method of treating cancer with a conjugate of any of formulae (I), (II) and (III) or composition thereof can be combined with still other methods of prophylactic and therapeutic treatment. Such methods include those that target destruction of cancer cells, e.g., by targeting of cell-surface markers, receptor ligands, e.g., ligands to gastrin-releasing peptide-like receptors, tumor-associated antigens, e.g., the 57 kD cytokeratin or the antigen recognized by the monoclonal antibody GB24, the extracellular matrix glycoprotein tamascin, antisense oncogenes such as c-fos, homeobox genes that are expressed in cancer cells but not normal cells, tumor-infiltrating lymphocytes that express cytokines, RGD-containing peptides and proteins, which are administered following surgery, lipophilic drug-containing liposomes to which are covalently conjugated monoclonal antibodies for targeting to cancer cells, low fat diet, moderate physical exercise and hormonal modulation. For prostate cancer, anti-testosterone agents can be used as well as an inhibitor of cellular proliferation produced by prostatic stromal cells and C-CAM, an epithelial cell adhesion molecule.

The conjugates of any of formulae (I), (II) and (III) are useful for targeting coding regions or control regions of genes, such as promoters or enhancers, and inhibiting transcription. Cancers that are suitable to treatment with conjugates of the present invention include those in which specific genes are known to be over-expressed and necessary for the survival of the cancer cell.

The following examples further illustrate the invention but, of course, should not be construed in any way as limiting its scope.

Examples for Formula (I):

### EXAMPLE 1

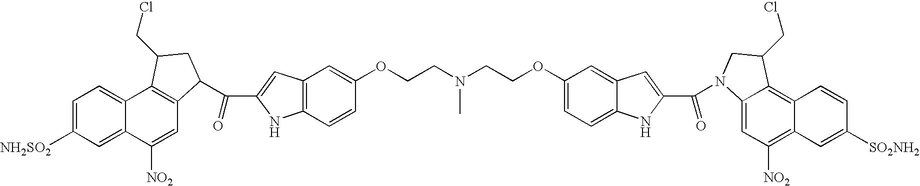 [0063] 3,3′-Bis-[2-(1-chloromethyl-5-nitro-7-sulfamoyl-1,2-dihydro-benzo[e]indole-3-carbonyl)-indole-5-yl]-N-Methyldiethanolamine,

### EXAMPLE 2

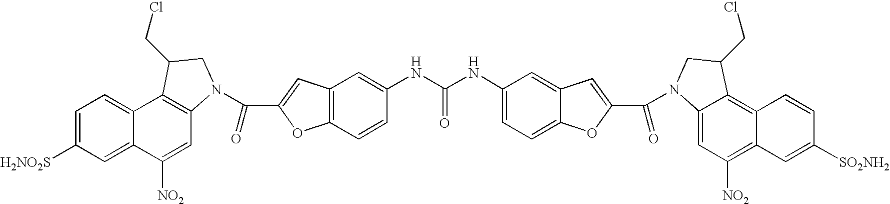 [0065] 1,3-Bis-[2-(1-chloromethyl-5-nitro-7-sulfamoyl-1,2-dihydro-benzo[e]indole-3-carbonyl)-benzofuran-5-yl]-urea,

### EXAMPLE 3

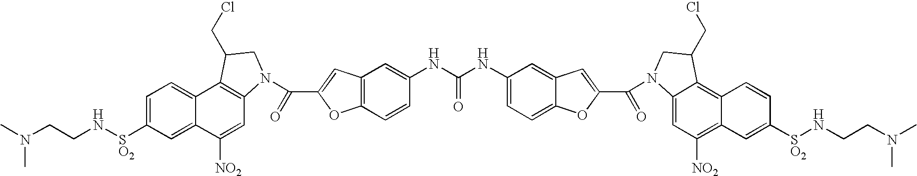 [0067] 1,3-Bis-{2-[1-chloromethyl-7-(2-dimethylamino-ethylsulfamoyl)-5-nitro-1,2-dihydro-benzo[e]indole-3-carbonyl]-benzofuran-5-yl}-urea,

### EXAMPLE 4

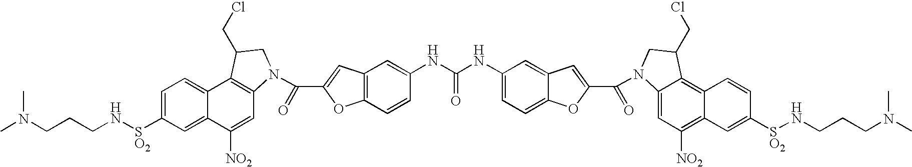 [0069] 1,3-Bis-{2-[1-chloromethyl-7-(3-dimethylamino-propylsulfamoyl)-5-nitro-1,2-dihydro-benzo[e]indole-3-carbonyl]-benzofuran-5-yl}-urea,

### EXAMPLE 5

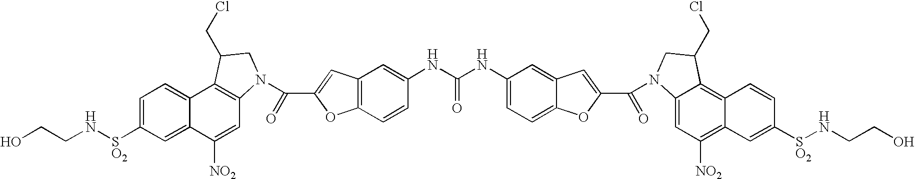 [0071] 1,3-Bis-{2-[1-chloromethyl-5-nitro-7-(2-hydroxy-ethylsulfamoyl)-1,2-dihydro-benzo[e]indole-3-carbonyl]-benzofuran-5-yl}-urea,

### EXAMPLE 6

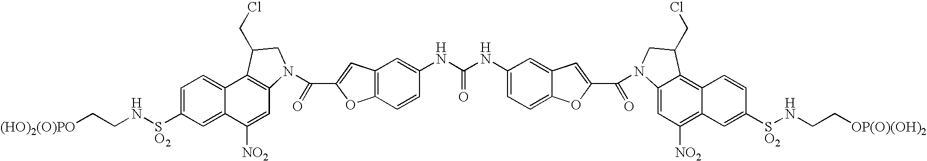 [0073] 1,3-Bis-{2-[1-chloromethyl-5-nitro-7-(2-phosphonooxy-ethylsulfamoyl)-1,2-dihydro-benzo[e]indole-3-carbonyl]-benzofuran-5-yl}-urea,

### EXAMPLE 7

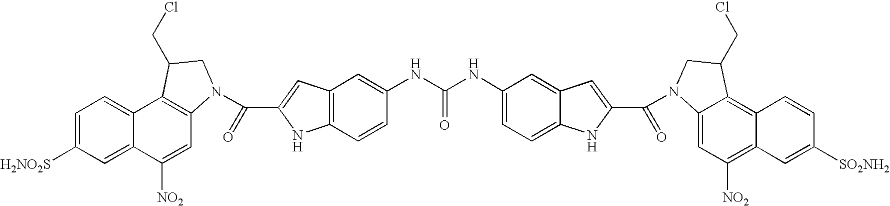 [0075] 1,3-Bis-[2-(1-chloromethyl-5-nitro-7-sulfamoyl-1,2-dihydro-benzo[e]indole-3-carbonyl)-1H-indol-5-yl]-urea,

### EXAMPLE 8

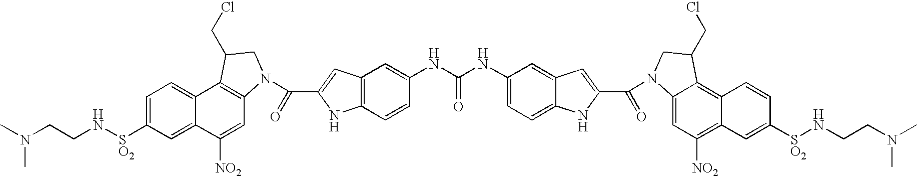 [0077] 1,3-Bis-{2-[1-chloromethyl-7-(2-dimethylamino-ethylsulfamoyl)-5-nitro-1,2-dihydro-benzo[e]indole-3-carbonyl]-1H-indol-5-yl}-urea,

### EXAMPLE 9

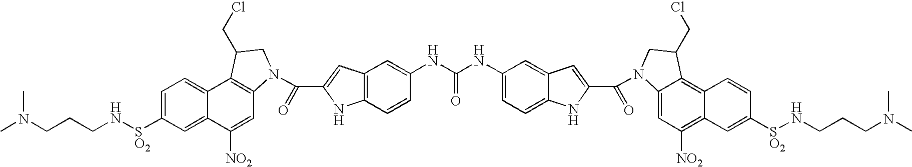 [0079] 1,3-Bis-{2-[1-chloromethyl-7-(3-dimethylamino-propylsulfamoyl)-5-nitro-1,2-dihydro-benzo[e]indole-3-carbonyl]-1H-indol-5-yl}-urea,

### EXAMPLE 10

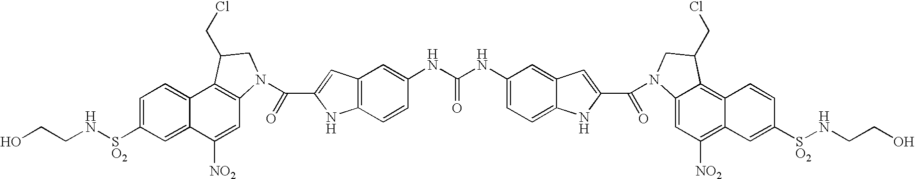 [0081] 1,3-Bis-{2-[1-chloromethyl-5-nitro-7-(2-hydroxy-ethylsulfamoyl)-1,2-dihydro-benzo[e]indole-3-carbonyl]-1H-indol-5-yl}-urea,

### EXAMPLE 11

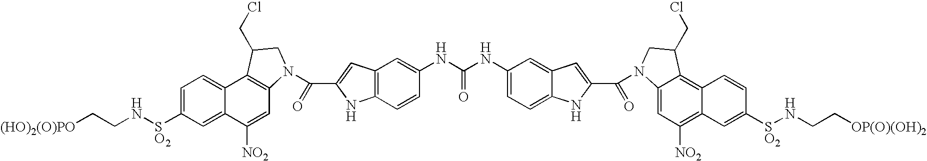 [0083] 1,3-Bis-{2-[1-chloromethyl-5-nitro-7-(2-phosphonooxy-ethylsulfamoyl)-1,2-dihydro-benzo[e]indole-3-carbonyl]-1H-indol-5-yl}-urea,

### EXAMPLE 12

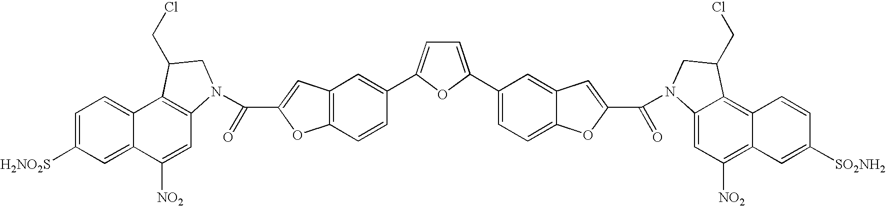 [0085] (5-{5-[2-(1-Chloromethyl-5-nitro-7-sulfamoyl-1,2-dihydro-benzo[e]indole-3-carbonyl)-benzofuran-5-yl]-furan-2-yl}-benzofuran-2-yl)-(1-chloromethyl-5-nitro-1,2-dihydro-benzo[e]indol-3-yl)-methanone,

### EXAMPLE 13

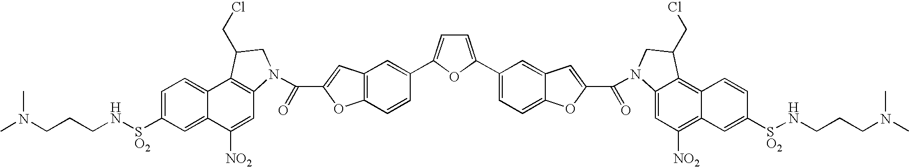 [0087] [5-(5-{2-[1-Chloromethyl-7-(3-dimethylamino-propylsulfamoyl)-5-nitro-1,2-dihydro-benzo[e]indole-3-carbonyl]-benzofuran-5-yl}-furan-2-yl)-benzofuran-2-yl]-(1-chloromethyl-7-(3-dimethylamino-propylsulfamoyl)-5-nitro-1,2-dihydro-benzo[e]indol-3-yl)-methanone,

### EXAMPLE 14

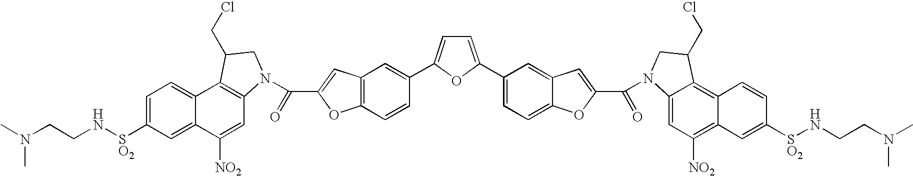 [0089] [5-(5-{2-[1-Chloromethyl-7-(2-dimethylamino-ethylsulfamoyl)-5-nitro-1,2-dihydro-benzo[e]indole-3-carbonyl]-benzofuran-5-yl}-furan-2-yl)-benzofuran-2-yl]-[1-chloromethyl-7-(2-dimethylamino-ethylsulfamoyl)-5-nitro-1,2-dihydro-benzo[e]indol-3-yl]-methanone,

### EXAMPLE 15

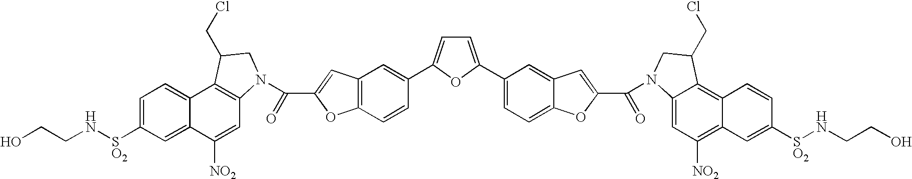 [0091] [5-(5-{2-[1-Chloromethyl-5-nitro-7-(2-hydroxy-ethylsulfamoyl)-1,2-dihydro-benzo[e]indole-3-carbonyl]-benzofuran-5-yl}-furan-2-yl)-benzofuran-2-yl]-[1-chloromethyl-7-(2-hydroxy-ethylsulfamoyl)-5-nitro-1,2-dihydro-benzo[e]indol-3-yl]-methanone,

### EXAMPLE 16

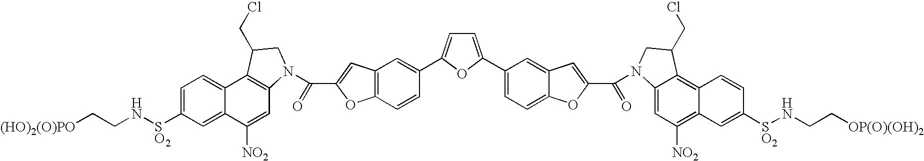 [0093] [5-(5-{2-[1-Chloromethyl-5-nitro-7-(2-phosphonooxy-ethylsulfamoyl)-1,2-dihydro-benzo[e]indole-3-carbonyl]-benzofuran-5-yl}-furan-2-yl)-benzofuran-2-yl]-(1-chloromethyl-5-nitro-7-(2-phosphonooxy-ethylsulfamoyl)-1,2-dihydro-benzo[e]indol-3-yl)-methanone,

### EXAMPLE 17

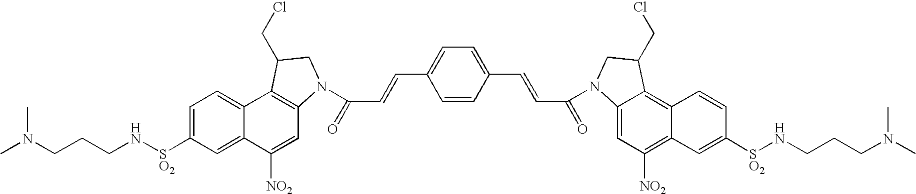 [0095] 1-[1-Chloromethyl-7-(3-dimethylamino-propylsulfamoyl)-5-nitro-1,2-dihydro-benzo[e]indol-3-yl)-3-(4-{3-[1-chloromethyl-7-(3-dimethylamino-propylsulfamoyl)-5-nitro-1,2-dihydro-benzo[e]indol-3-yl]-3-oxo-propenyl}-phenyl)-propenone,

### EXAMPLE 18

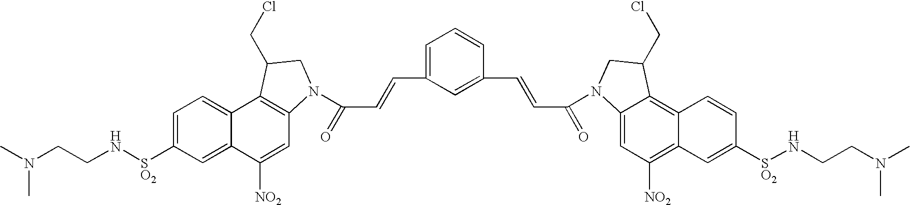 [0097] 1-[1-Chloromethyl-7-(2-dimethylamino-ethylsulfamoyl)-5-nitro-1,2-dihydro-benzo[e]indol-3-yl]-3-(3-{3-[1-chloromethyl-7-(2-dimethylamino-ethylsulfamoyl)-5-nitro-1,2-dihydro-benzo[e]indol-3-yl]-3-oxo-propenyl}-phenyl)-propenone,

### EXAMPLE 19

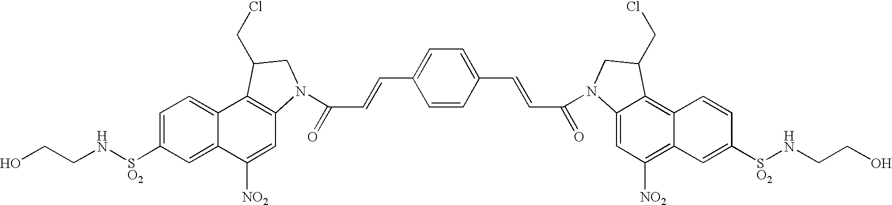 [0099] 1-[1-Chloromethyl-7-(2-hydroxy-ethylsulfamoyl)-5-nitro-1,2-dihydro-benzo[e]indol-3-yl)-3-(4-{3-[1-chloromethyl-7-(2-hydroxy-ethylsulfamoyl)-5-nitro-1,2-dihydro-benzo[e]indol-3-yl]-3-oxo-propenyl}-phenyl)-propenone,

### EXAMPLE 20

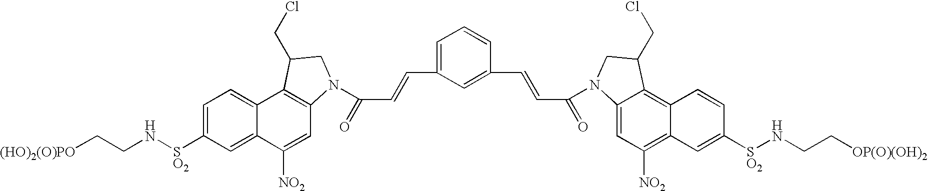 [0101] 1-[1-Chloromethyl-7-(2-phosphonooxy-ethylsulfamoyl)-5-nitro-1,2-dihydro-benzo[e]indol-3-yl]-3-(3-{3-[1-chloromethyl-7-(2-phosphonooxy-ethylsulfamoyl)-5-nitro-1,2-dihydro-benzo[e]indol-3-yl]-3-oxo-propenyl}-phenyl)-propenone,

### EXAMPLE 21

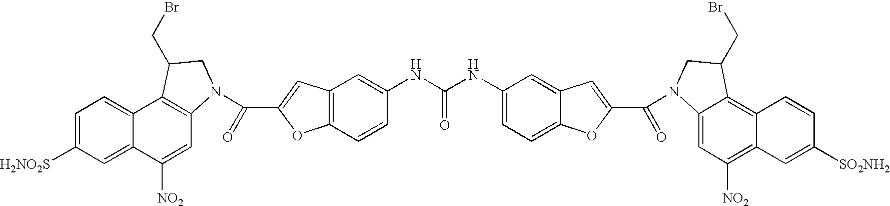 [0103] 1,3-Bis-[2-(1-bromomethyl-5-nitro-7-sulfamoyl-1,2-dihydro-benzo[e]indole-3-carbonyl)-benzofuran-5-yl]-urea,

### EXAMPLE 22

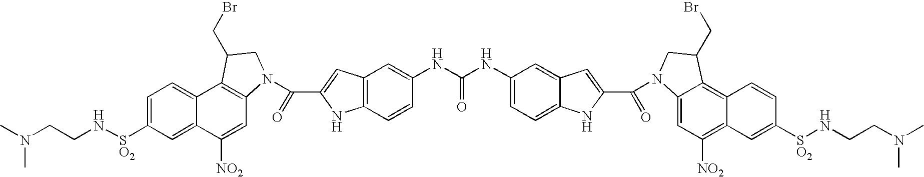 [0105] 1,3-Bis-{2-[1-bromomethyl-7-(2-dimethylamino-ethylsulfamoyl)-5-nitro-1,2-dihydro-benzo[e]indole-3-carbonyl]-1H-indol-5-yl}-urea,

### EXAMPLE 23

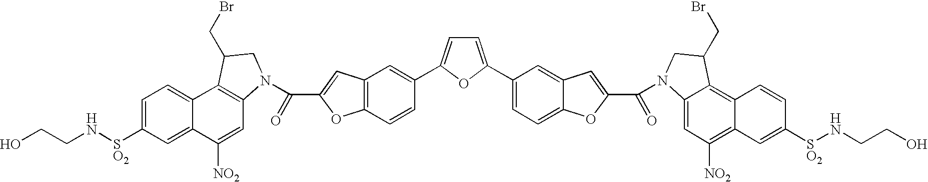 [0107] [5-(5-{2-[1-Bromomethyl-5-nitro-7-(2-hydroxy-ethylsulfamoyl)-1,2-dihydro-benzo[e]indole-3-carbonyl]-benzofuran-5-yl}-furan-2-yl)-benzofuran-2-yl]-[1-bromomethyl-7-(2-hydroxy-ethylsulfamoyl)-5-nitro-1,2-dihydro-benzo[e]indol-3-yl]-methanone,

### EXAMPLE 24

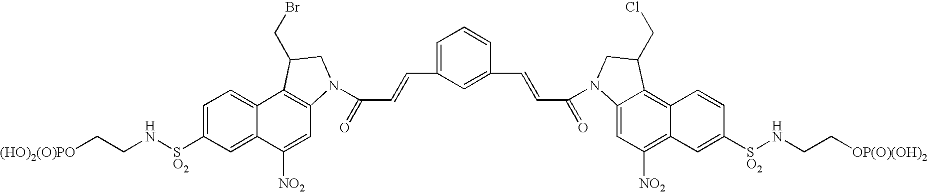 [0109] 1-[1-Bromomethyl-7-(2-phosphonooxy-ethylsulfamoyl)-5-nitro-1,2-dihydro-benzo[e]indol-3-yl]-3-(3-{3-[1-chloromethyl-7-(2-phosphonooxy-ethylsulfamoyl)-5-nitro-1,2-dihydro-benzo[e]indol-3-yl]-3-oxo-propenyl}-phenyl)-propenone.

Examples for Formula (II):

### EXAMPLE 25

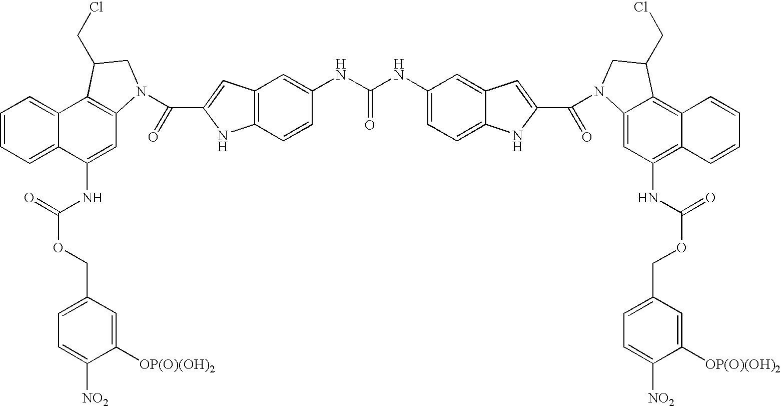 [0112] 1,3-Bis-{2-[1-chloromethyl-5-(3-dihydrogenphosphonoxy-4-nitrobenzyloxycarbonyl-amino)-1,2-dihydro-benzo[e]indole-3-carbonyl]-1H-indol-5-yl}-urea.

### EXAMPLE 26

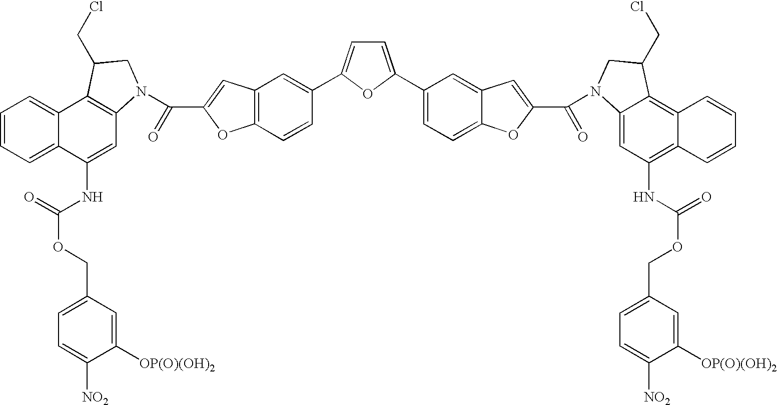 [0114] [5-(5-{2-[1-chloromethyl-5-(3-dihydrogenphosphonoxy-4-nitrobenzyloxycarbonyl-amino)-1,2-dihydro-benzo[e]indole-3-carbonyl]-benzofuran-5-yl}-furan-2-yl)-benzofuran-2-yl]-[1-chloromethyl-5-(3-dihydrogenphosphonoxy-4-nitrobenzyloxycarbonyl-amino)-1,2-dihydro-benzo[e]indol-3-yl]-methanone,

### EXAMPLE 27

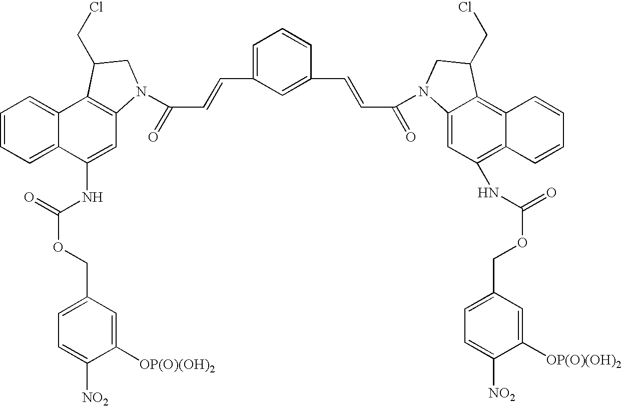 [0116] 1-(5-(3-dihydrogenphosphonoxy-4-nitrobenzyloxycarbonyl-amino)-1-chloromethyl-1,2-dihydro-benzo[e]indol-3-yl)-3-(3-{3-[5-(3-dihydrogenphosphonoxy-4-nitrobenzyloxycarbonyl-amino)-1-chloromethyl-1,2-dihydro-benzo[e]indol-3-yl]-3-oxo-propenyl}-phenyl)-propenone,

### EXAMPLE 28

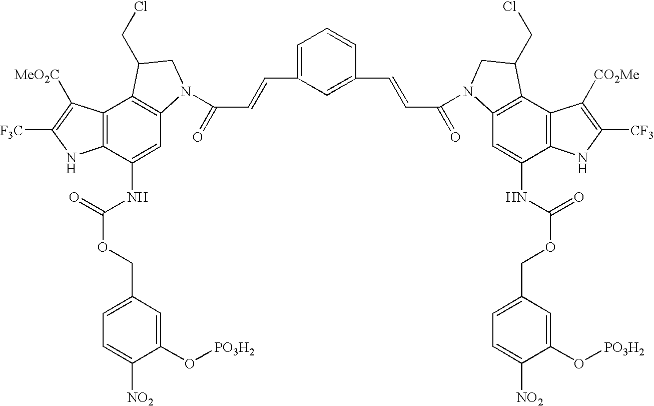 [0118] 1-[1-chloromethyl-5-(3-dihydrogenphosphonoxy-4-nitrobenzyloxycarbonyl-amino)-8-methoxycarbonyl-7-trifluoromethyl-1,6-dihydro-2H-3,6-diaza-as-indacen-3-yl]-3-{3-[3-(1-chloromethyl-5-(3-dihydrogenphosphonoxy-4-nitrobenzyloxycarbonyl-amino)-8-methoxycarbonyl-7-trifluoromethyl-1,6-dihydro-2H-3,6-diaza-as-indacen-3-yl)-3-oxo-propenyl]-phenyl}-propenone.

Example for Formula (III):

### EXAMPLE 29

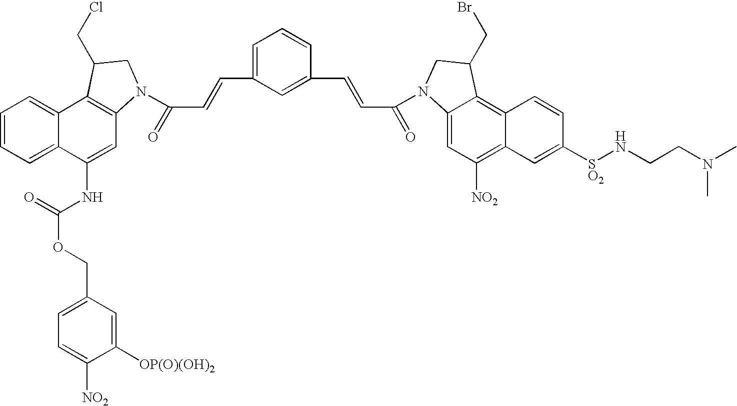 [0121] 1-[5-(3-dihydrogenphosphonoxy-4-nitrobenzyloxycarbonyl-amino)-1-chloromethyl-1,2-dihydro-benzo[e]indol-3-yl]-3-(3-{3-[1-bromomethyl-7-(2-dimethylamino-ethylsulfamoyl)-5-nitro-1,2-dihydro-benzo[e]indol-3-yl]-3-oxo-propenyl}-phenyl)-propenone.

Chemical Procedures:

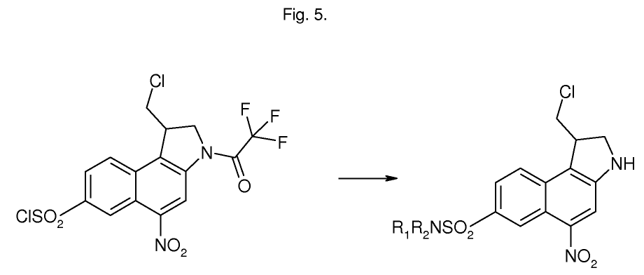

### 1-(chloromethyl)-5-nitro-1,2-dihydro-3H-benzo[e]indole-7sulfonamide (FIG. 5.)

1-(chloromethyl)-5-nitro-3-(trifluoroacetyl)-1,2-dihydro-3H-benzo[e]indole-7-sulfonyl chloride (20 mg, 0.054 mM) was dissolved in THF (1.5 mL) at room temperature. The solution was cooled to 0° C. and treated with conc. ammonia (50 μL) and stirred for 15 min while the solution was warmed up to room temperature and conc. ammonia was added (50 μL) and the stirring was continued for another 30 min, until the completion was confirmed by TLC analysis. The reaction mixture was diluted with water (30 mL) and extracted with dichloromethane (3×15 mL). The combined organic phase was washed with brine, dried with sodium sulfate, filtered, treated with methanolic hydrogen chloride and evaporated under reduced pressure.

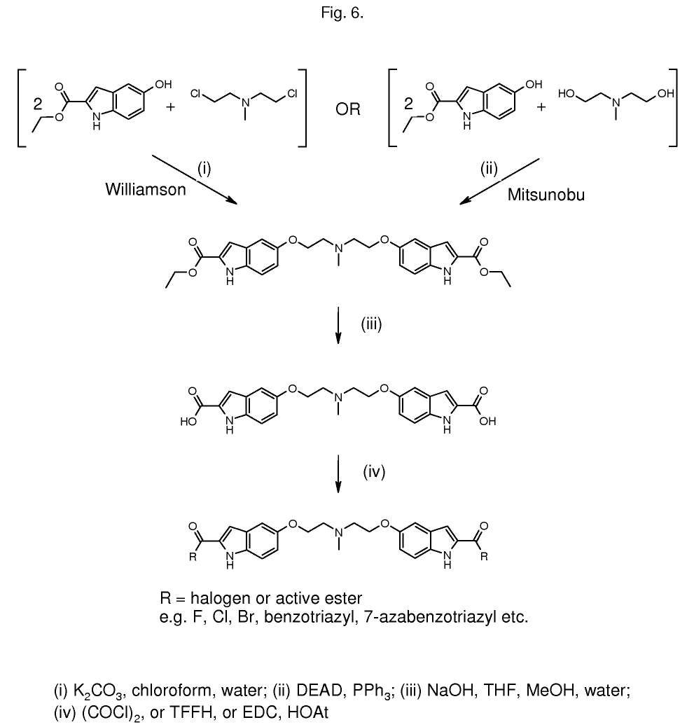

In order to synthesize highly effective bis-alkylating agents (containing either one or two CBN units), the selection of the linker is an important issue. Linkers containing two 2-carboxy-5-hydroxy-indole units are the most desirable. Their indole building block is the commercially available Ethyl 5-hydroxy-indole-2-carboxylate. Two distinct synthetic approaches were explored, namely the Williamson and the Mitsunobu protocols (FIG. 6). Besides the indole derivative, for the Williamson ether synthesis the second reactant is mechloretamine hydrochloride and for the Mitsunobu reaction the second reactant is the N-Methyldiethanolamine.

N,N-Bis((2-Ethylcarboxyl-indole-5-yl)ethoxy)-N-methylamine and its activation (FIG. 6.) Mechoretamine and ethyl-5-hydroxyindole-2-carboxylate were dissolved in chloroform water mixture (4:1) (ml). Potassium carbonate was added and the mixture was refluxed for 10 hrs. The reaction mixture was diluted with water and extracted with chloroform. The combined organic phase was dried and evaporated. The product was purified by column chromatography (ethyl acetate gradient in hexane).

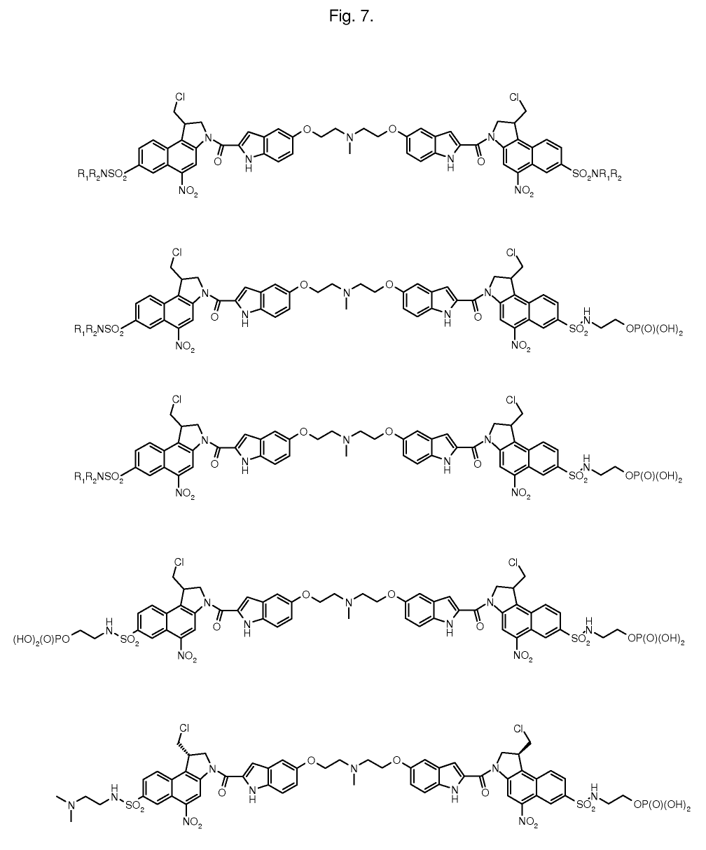

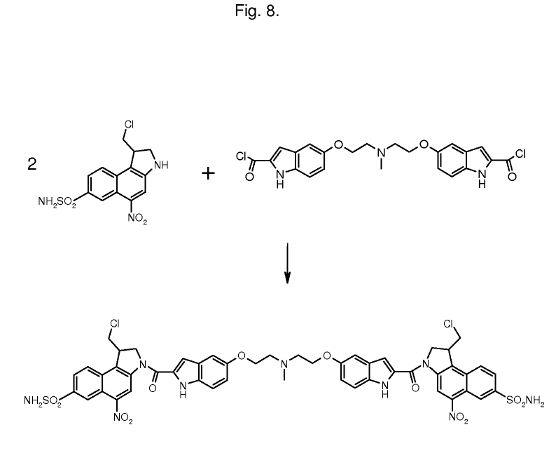

In order to synthesize various CBN dimmers (FIG. 7), the activated di-carboxylic acid linker was coupled to the CBN units by using regular conditions (FIG. 8).

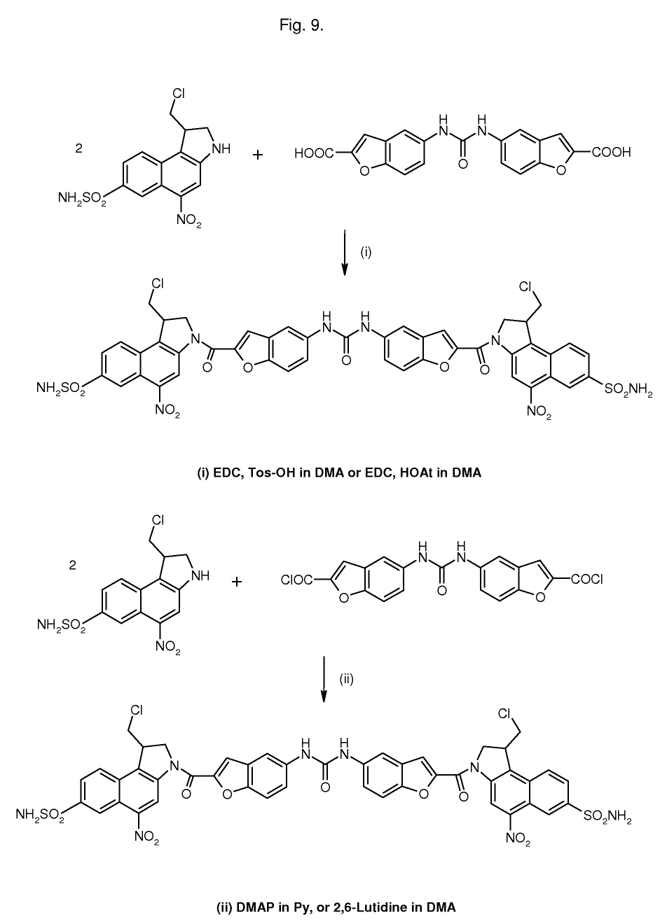

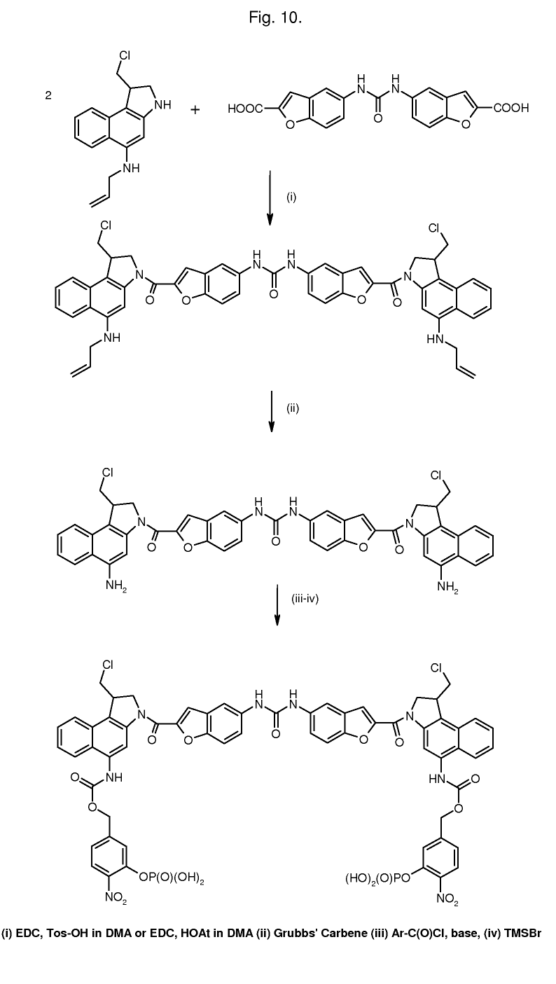

Various other conditions are used as described in the art, like coupling with carbonyl chlorides or active esters to produce CBN and CBA containing bis-alkylating agents with the proper linkers (FIG. 9 and FIG. 10)

1. We claim a compound of formula I for treating cancer in a mammal, 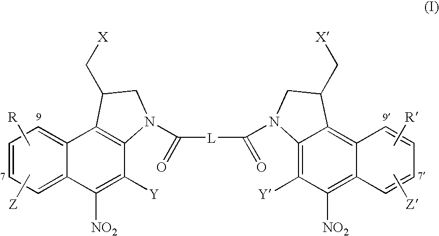
wherein X and X′ are independently selected from halogen (preferably chlorine or bromine),
wherein R, R′, Y, Y′, Z and Z′ are independently selected from hydrogen, halogen, C₁₋₄ alkyl, OR¹, OP(O)(OH)₂, NR¹C(O)OR², OC(O)R¹, NHC(O)NR¹R², SR¹, NR¹₂, COR¹, SOR¹, SO₂R¹, SO₂NH₂, SO₂NR¹OR², SO₂NR¹NR², SO₂NHCOR¹, CO₂R¹, CONR¹₂, CONHSO₂R¹, CF₃, CN, NO₂, where R¹ and R² represent hydrogen or C₁₋₄ alkyl, where R, R′, Z and Z′ are located at any of the available positions 6-9 or located at any of the available positions of the A and A′ ring systems and R, R′, Z and Z′ independently represent a C₁₋₄ alky group or a SO₂NHR¹ group optionally further substituted with one or more amino, thiol, hydroxyl groups, each amino or thiol group being further optionally substituted with one or two C₁₋₄ alkyl groups and each hydroxyl group being further optionally substituted with a phosphate group,
and wherein L represent a linker selected from but not limited to: urea, carbamate, ethylene glycol, propylene glycol, unfused aromatic, fused aromatic, hetero-aromatic, 1,4-phenylenediacryloyl, 1,3-phenylenediacryloyl building blocks, with a special interest in linked indoles like N,N-Bis[indole-5-yl-(2-hydroxyethyl)]methylamine.

2. We claim a compound of Formula II for treating cancer in a mammal, 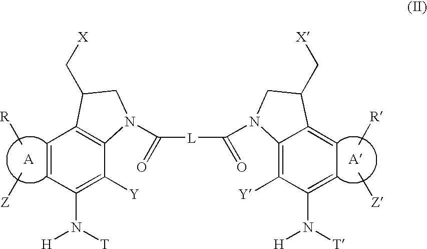
wherein X and X′ are independently selected from halogen (preferably chlorine or bromine),
wherein R, R′, Y, Y′, Z and Z′ are independently selected from hydrogen, halogen, C₁₋₄ alkyl, OR¹, OP(O)(OH)₂, NR¹C(O)OR², OC(O)R¹, NHC(O)NR¹R², SR¹, NR¹₂, COR¹, SOR¹, SO₂R¹, SO₂NH₂, SO₂NR¹OR², SO₂NR¹NR²₃, SO₂NHCOR¹, CO₂R¹, CONR¹₂, CONHSO₂R¹, CF₃, CN, NO₂, where R¹ and R² represent hydrogen or C₁₋₄ alkyl, where R, R′, Z and Z′ are located at any of the available positions 6-9 or located at any of the available positions of the A and A′ ring systems and R, R′, Z and Z′ independently represent a C₁₋₄ alky group or a SO₂NHR¹ group optionally further substituted with one or more amino, thiol, hydroxyl groups, each amino or thiol group being further optionally substituted with one or two C₁₋₄ alkyl groups and each hydroxyl group being further optionally substituted with a phosphate group,
wherein A and A′ are independently selected aromatic, heteroaromatic or saturated carbocyclic or heterocyclic systems (preferably one of the following rings: benzene, pyridine, imidazole, pyrrole, the saturated part of teralin), or A and A′ independently represent two hydrogen atoms,
wherein L represent a linker selected from but not limited to: urea, carbamate, ethylene glycol, propylene glycol, unfused aromatic, fused aromatic, hetero-aromatic, 1,4-phenylenediacryloyl, 1,3-phenylenediacryloyl building blocks,
and wherein T and T′ represent hypoxia-sensitive groups, independently selected from nitro-group containing aromatic groups (e.g. 4-nitro-benzyl) with optional substituents (like one or more amino, thiol, hydroxyl groups), with optional further substituents (like phosphate or C₁₋₄ alkyl groups).

3. We claim a compound of Formula III for treating cancer in a mammal, 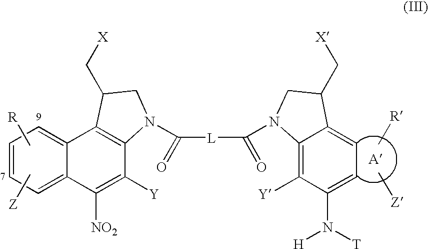
wherein X and X′ are independently selected from halogen (preferably chlorine or bromine),
wherein R, R′, Y, Y′, Z and Z′ are independently selected from hydrogen, halogen, C₁₋₄ alkyl, OR¹, OP(O)(OH)₂, NR¹C(O)OR², OC(O)R¹, NHC(O)NR¹R², SR¹, NR¹₂, COR¹, SOR¹, SO₂R¹, SO₂NH₂, SO₂NR¹OR², SO₂NR¹NR²₃, SO₂NHCOR¹, CO₂R¹, CONR¹₂, CONHSO₂R¹, CF₃, CN, NO₂, where R¹ and R² represent hydrogen or C₁₋₄ alkyl, where R, R′, Z and Z′ are located at any of the available positions 6-9 or located at any of the available positions of the A ring system and R, R′, Z and Z′ independently represent a C₁₋₄ alky group or a SO₂NHR¹ group optionally further substituted with one or more amino, thiol, hydroxyl groups, each amino or thiol group being further optionally substituted with one or two C₁₋₄ alkyl groups and each hydroxyl group being further optionally substituted with a phosphate group,
wherein A is independently selected from aromatic, heteroaromatic or saturated carbocyclic or heterocyclic systems (preferably one of the following rings: benzene, pyridine, imidazole, pyrrole, the saturated part of teralin), or A represents two hydrogen atoms,
wherein L represent a linker selected from but not limited to: urea, carbamate, ethylene glycol, propylene glycol, unfused aromatic, fused aromatic, hetero-aromatic, 1,4-phenylenediacryloyl, 1,3-phenylenediacryloyl building blocks, with a special interest in indole containing moieties like 5-hydroxy-indole-2-carboxylic acid attached to the CBN unit,
and wherein T represents a hypoxia-sensitive group, selected from nitro-group containing aromatic groups (e.g. 4-nitro-benzyl) with optional substituents (like one or more amino, thiol, hydroxyl groups), with optional further substituents (like phosphate or C₁₋₄ alkyl groups).

4. We claim the use of Formula (I) in ADEPT therapy.

5. We claim the use of Formula (I) in GDEPT therapy.

6. We claim the use of Formula (II) in ADEPT therapy.

7. We claim the use of Formula (II) in GDEPT therapy.

8. We claim the use of Formula (III) in ADEPT therapy.

9. We claim the use of Formula (III) in GDEPT therapy.

10. The invention claims the methods as claimed in any one of claims 1 to 9 in connection with radiotherapy, administering any of the Formulae before, during or after the radiation.

11. We claim a compound of Formula (I) as claimed in claim 1 selected from, but not limited to the following:
3,3′-Bis-[2-(1-chloromethyl-5-nitro-7-sulfamoyl-1,2-dihydro-benzo[e]indole-3-carbonyl)-indole-5-yl]-N-Methyldiethanolamine,
1,3-Bis-[2-(1-chloromethyl-5-nitro-7-sulfamoyl-1,2-dihydro-benzo[e]indole-3-carbonyl)benzofuran-5-yl]-urea,
1,3-Bis-{2-[1-chloromethyl-7-(2-dimethylamino-ethylsulfamoyl)-5-nitro-1,2-dihydro-benzo[e]indole-3-carbonyl]-benzofuran-5-yl}-urea,
1,3-Bis-{2-[1-chloromethyl-7-(3-dimethylamino-propylsulfamoyl)-5-nitro-1,2-dihydro-benzo[e]indole-3-carbonyl]-benzofuran-5-yl}-urea,
1,3-Bis-{2-[1-chloromethyl-5-nitro-7-(2-hydroxy-ethylsulfamoyl)-1,2-dihydro-benzo[e]indole-3-carbonyl]-benzofuran-5-yl}-urea,
1,3-Bis-{2-[1-chloromethyl-5-nitro-7-(2-phosphonooxy-ethylsulfamoyl)-1,2-dihydro-benzo[e]indole-3-carbonyl]-benzofuran-5-yl}-urea,
1,3-Bis-[2-(1-chloromethyl-5-nitro-7-sulfamoyl-1,2-dihydro-benzo[e]indole-3-carbonyl)-1H-indol-5-yl]-urea,
1,3-Bis-{2-[1-chloromethyl-7-(2-dimethylamino-ethylsulfamoyl)-5-nitro-1,2-dihydro-benzo[e]indole-3-carbonyl]-1H-indol-5-yl}-urea,
1,3-Bis-{2-[1-chloromethyl-7-(3-dimethylamino-propylsulfamoyl)-5-nitro-1,2-dihydro-benzo[e]indole-3-carbonyl]-1H-indol-5-yl}-urea,
1,3-Bis-{2-[1-chloromethyl-5-nitro-7-(2-hydroxy-ethylsulfamoyl)-1,2-dihydro-benzo[e]indole-3-carbonyl]-1H-indol-5-yl}-urea,
1,3-Bis-{2-[1-chloromethyl-5-nitro-7-(2-phosphonooxy-ethylsulfamoyl)-1,2-dihydro-benzo[e]indole-3-carbonyl]-1H-indol-5-yl}-urea,
(5-{5-[2-(1-Chloromethyl-5-nitro-7-sulfamoyl-1,2-dihydro-benzo[e]indole-3-carbonyl)benzofuran-5-yl]-furan-2-yl}-benzofuran-2-yl)-(1-chloromethyl-5-nitro-1,2-dihydro-benzo[e]indol-3-yl)-methanone,
[5-(5-{2-[1-Chloromethyl-7-(3-dimethylamino-propylsulfamoyl)-5-nitro-1,2-dihydro-benzo[e]indole-3-carbonyl]-benzofuran-5-yl}-furan-2-yl)-benzofuran-2-yl]-(1-chloromethyl-7-(3-dimethylamino-propylsulfamoyl)-5-nitro-1,2-dihydro-benzo[e]indol-3-yl)-methanone,
[5-(5-{2-[1-Chloromethyl-7-(2-dimethylamino-ethylsulfamoyl)-5-nitro-1,2-dihydro-benzo[e]indole-3-carbonyl]-benzofuran-5-yl}-furan-2-yl)-benzofuran-2-yl]-[1-chloromethyl-7-(2-dimethylamino-ethylsulfamoyl)-5-nitro-1,2-dihydro-benzo[e]indol-3-yl]-methanone,
[5-(5-{2-[1-Chloromethyl-5-nitro-7-(2-hydroxy-ethylsulfamoyl)-1,2-dihydro-benzo[e]indole-3-carbonyl]-benzofuran-5-yl}-furan-2-yl)-benzofuran-2-yl]-[1-chloromethyl-7-(2-hydroxy-ethylsulfamoyl)-5-nitro-1,2-dihydro-benzo[e]indol-3-yl]-methanone,
[5-(5-{2-[1-Chloromethyl-5-nitro-7-(2-phosphonooxy-ethylsulfamoyl)-1,2-dihydro-benzo[e]indole-3-carbonyl]-benzofuran-5-yl}-furan-2-yl)-benzofuran-2-yl]-(1-chloromethyl-5-nitro-7-(2-phosphonooxy-ethylsulfamoyl)-1,2-dihydro-benzo[e]indol-3-yl)-methanone,
1-[1-Chloromethyl-7-(3-dimethylamino-propylsulfamoyl)-5-nitro-1,2-dihydro-benzo[e]indol-3-yl)-3-(4-{3-[1-chloromethyl-7-(3-dimethylamino-propylsulfamoyl)-5-nitro-1,2-dihydro-benzo[e]indol-3-yl]-3-oxo-propenyl}-phenyl)-propenone,
1-[1-Chloromethyl-7-(2-dimethylamino-ethylsulfamoyl)-5-nitro-1,2-dihydro-benzo[e]indol-3-yl]-3-(3-{3-[1-chloromethyl-7-(2-dimethylamino-ethylsulfamoyl)-5-nitro-1,2-dihydro-benzo[e]indol-3-yl]-3-oxo-propenyl}-phenyl)-propenone,
1-[1-Chloromethyl-7-(2-hydroxy-ethylsulfamoyl)-5-nitro-1,2-dihydro-benzo[e]indol-3-yl)-3-(4-{3-[1-chloromethyl-7-(2-hydroxy-ethylsulfamoyl)-5-nitro-1,2-dihydro-benzo[e]indol-3-yl]-3-oxo-propenyl}-phenyl)-propenone,
1-[1-Chloromethyl-7-(2-phosphonooxy-ethylsulfamoyl)-5-nitro-1,2-dihydro-benzo[e]indol-3-yl]-3-(3-{3-[1-chloromethyl-7-(2-phosphonooxy-ethylsulfamoyl)-5-nitro-1,2-dihydro-benzo[e]indol-3-yl]-3-oxo-propenyl}-phenyl)-propenone,
1,3-Bis-[2-(1-bromomethyl-5-nitro-7-sulfamoyl-1,2-dihydro-benzo[e]indole-3-carbonyl)-benzofuran-5-yl]-urea,
1,3-Bis-{2-[1-bromomethyl-7-(2-dimethylamino-ethylsulfamoyl)-5-nitro-1,2-dihydro-benzo[e]indole-3-carbonyl]-1H-indol-5-yl}-urea,
[5-(5-{2-[1-Bromomethyl-5-nitro-7-(2-hydroxy-ethylsulfamoyl)-1,2-dihydro-benzo[e]indole-3-carbonyl]-benzofuran-5-yl}-furan-2-yl)-benzofuran-2-yl]-[1-bromomethyl-7-(2-hydroxy-ethylsulfamoyl)-5-nitro-1,2-dihydro-benzo[e]indol-3-yl]-methanone,
1-[1-Bromomethyl-7-(2-phosphonooxy-ethylsulfamoyl)-5-nitro-1,2-dihydro-benzo[e]indol-3-yl]-3-(3-{3-[1-chloromethyl-7-(2-phosphonooxy-ethylsulfamoyl)-5-nitro-1,2-dihydro-benzo[e]indol-3-yl]-3-oxo-propenyl}-phenyl)-propenone.

12. We claim a compound of Formula (II) as claimed in claim 2 selected from, but not limited to the following:
1,3-Bis-{2-[1-chloromethyl-5-(3-dihydrogenphosphonoxy-4-nitrobenzyloxycarbonyl-amino)-1,2-dihydro-benzo[e]indole-3-carbonyl]-1H-indol-5-yl}-urea,
[5-(5-{2-[1-chloromethyl-5-(3-dihydrogenphosphonoxy-4-nitrobenzyloxycarbonyl-amino)-1,2-dihydro-benzo[e]indole-3-carbonyl]-benzofuran-5-yl}-furan-2-yl)-benzofuran-2-yl]-[1-chloromethyl-5-(3-dihydrogenphosphonoxy-4-nitrobenzyloxycarbonyl-amino)-1,2-dihydro-benzo[e]indol-3-yl]-methanone,
1-(5-(3-dihydrogenphosphonoxy-4-nitrobenzyloxycarbonyl-amino)-1-chloromethyl-1,2-dihydro-benzo[e]indol-3-yl)-3-(3-{3-[5-(3-dihydrogenphosphonoxy-4-nitrobenzyloxycarbonyl-amino)-1-chloromethyl-1,2-dihydro-benzo[e]indol-3-yl]-3-oxo-propenyl}-phenyl)-propenone,
1-[5-(3-dihydrogenphosphonoxy-4-nitrobenzyloxycarbonyl-amino)-1-chloromethyl-1,2-dihydro-benzo[e]indol-3-yl]-3-(3-{3-[1-bromomethyl-7-(2-dimethylamino-ethylsulfamoyl)-5-nitro-1,2-dihydro-benzo[e]indol-3-yl]-3-oxo-propenyl}-phenyl)-propenone,
1-[1-chloromethyl-5-(3-dihydrogenphosphonoxy-4-nitrobenzyloxycarbonyl-amino)-8-methoxycarbonyl-7-trifluoromethyl-1,6-dihydro-2H-3,6-diaza-as-indacen-3-yl]-3-{3-[3-(1-chloromethyl-5-(3-dihydrogenphosphonoxy-4-nitrobenzyloxycarbonyl-amino)-8-methoxycarbonyl-7-trifluoromethyl-1,6-dihydro-2H-3,6-diaza-as-indacen-3-yl)-3-oxo-propenyl]-phenyl}-propenone.

13. We claim a compound of Formula (III) as claimed in claim 3 represented by, but not limited to the following:
1-[5-(3-dihydrogenphosphonoxy-4-nitrobenzyloxycarbonyl-amino)-1-chloromethyl-1,2-dihydro-benzo[e]indol-3-yl]-3-(3-{3-[1-bromomethyl-7-(2-dimethylamino-ethylsulfamoyl)-5-nitro-1,2-dihydro-benzo[e]indol-3-yl]-3-oxo-propenyl}-phenyl)-propenone.

14. We claim the use of any optical isomer of Formula (I) and their mixtures for use to treat cancer in mammals.

15. We claim the use of any optical isomer of Formula (II) and their mixtures for use to treat cancer in mammals.

16. We claim the use of any optical isomer of Formula (III) and their mixtures for use to treat cancer in mammals.

17. We claim the use of a compound of Formula (I) as claimed in claim 1 in chemotherapy combined with one or more chemicals. This includes but is not limited to check point abrogators and analogues there of.

18. We claim the use of a compound of Formula (II) as claimed in claim 2 in chemotherapy combined with one or more chemicals. This includes but is not limited to check point abrogators and analogues there of.

19. We claim the use of a compound of Formula (III) as claimed in claim 3 in chemotherapy combined with one or more chemicals. This includes but is not limited to check point abrogators and analogues there of.

20. We claim the use of a compound of Formula (I) as claimed in claim 1 in chemotherapy combined with one or more biologicals.

21. We claim the use of a compound of Formula (II) as claimed in claim 2 in chemotherapy combined with one or more biologicals.

22. We claim the use of a compound of Formula (III) as claimed in claim 3 in chemotherapy combined with one or more biologicals.

23. We claim the use of a compound of Formula (I) as claimed in claim 1 in cancer therapy combined with one or more biologicals and chemicals.

24. We claim the use of a compound of Formula (II) as claimed in claim 2 in chemotherapy combined with one or more biologicals and chemicals.

25. We claim the use of a compound of Formula (III) as claimed in claim 3 in chemotherapy combined with one or more biologicals and chemicals.

26. We claim the use of an excessive amount of a base (e.g. potassium carbonate), two equivalent of 5-hydroxy-indole derivative and relative to the latter one equivalent of mechloretamine hydrochloride to achieve a high yielding synthesis of the indole based linkers.

27. We claim the use of a triphenylphosphine as well as an azodicarboxilic compound (e.g. diethylazodicarboxylate) for the synthesis of the indole based linkers starting from a 5-hydroxy-indole derivative and an alcohol (e.g. N-Methyldiethanolamine) at 2:1 ratio.
